(19) United States
(12) Patent Application Publication
(10) Pub. No.: US 2024/0009429 A1
(43) Pub. Date: Jan. 11, 2024

(54) Guidewire

(71) Applicant: Micro-Tech Endoscopy USA Inc., Ann Arbor, MI (US)

(72) Inventors: Brian Lim, Irvine, CA (US); Xu Chao Wei, Nanjing (CN); Ren Gang, Nanjing (CN)

(21) Appl. No.: 17/857,268

(22) Filed: Jul. 5, 2022

(51) Int. Cl. A61M 25/09

(52) U.S. Cl. CPC A61M 25/09; A61M 2025/09108; A61M 2025/09083; A61M 2025/09091

(57) ABSTRACT

A guidewire includes a first wire with a first diameter. A second wire is coupled with the first wire and has a second diameter that is smaller than the first diameter. A winding extends around the second wire to cover a portion of the second wire and leaves a remainder of the second wire uncovered.

### CROSS-REFERENCE TO RELATED APPLICATION(S)

None.

### TECHNICAL FIELD

This disclosure relates to endoscopy and devices for navigating gastrointestinal structures within a human body.

### BACKGROUND

A guidewire is a device used to navigate through gastrointestinal structures (e.g., bile and/or pancreatic ducts, esophagus strictures, etc.) to reach a target site within the body. In many instances the guidewire must navigate a narrow and/or tortuous path in the gastrointestinal system (e.g., the pancreatobiliary system). The tortuous path may require the guidewire to bend, twist, turn, or otherwise be positioned to follow one or more branches of ducts to be located deep into the gastrointestinal system (e.g., the biliary or pancreatic systems). Upon achieving deep cannulation of the bile/pancreatic duct, the guidewire acts as a guide that another device (e.g., a catheter) can follow to perform the required diagnostic procedure (e.g., biopsy) and/or the treatment (e.g., stenting) steps in the procedure. Without a guidewire, pancreatobiliary endoscopy is not possible.

Guidewires are typically characterized by their pushability, steerability, torque and opacity. Pushability is the amount of force needed to advance the wire. Steerability is the ability and responsiveness of the wire tip to navigate gastrointestinal structures. Torque is the response of the wire to turning by the operator when navigating gastrointestinal structures. Its opacity is its level of visibility under fluoroscopic imaging.

### SUMMARY

In one embodiment, a wire includes an outer surface that defines a diameter. A winding extends around the wire and defines an undulating surface that extends along a length of the wire. A peak of the undulating surface is defined by the winding and a valley of the undulating surface is defined by the outer surface of the wire.

In another embodiment, a guidewire includes a wire with an outer surface that defines a diameter and a recess extending into and helically around the wire. A winding is configured to fit within the recess and extend helically around the wire.

In yet another embodiment, a method of producing a guidewire includes coupling a first end of a winding to a wire, wrapping the winding around the wire, and coupling a second end of the winding to the wire.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 is a diagram of a guidewire, according to an exemplary embodiment.

FIG. 2 is a diagram of a cross-section of the guidewire of FIG. 1.

FIG. 3 is another diagram of a cross-section of the guidewire of FIG. 1.

FIG. 4 is a diagram of a winding of the guidewire of FIG. 1.

FIG. 5 is a diagram of another winding, according to an exemplary embodiment.

FIGS. 6-7 are diagrams of another guidewire, according to an exemplary embodiment.

FIG. 8 is a diagram of a winding of the guidewire of FIG. 7.

FIG. 9 is a flow chart of a process to manufacture a guidewire, according to an exemplary embodiment.

### DETAILED DESCRIPTION

Disclosed herein are devices and methods for navigating a tortuous path through gastrointestinal structures of a patient to reach a target site. The devices and methods, more specifically, include a guidewire wrapped with a winding. The winding extends around a circumference of the guidewire and extends along a length of the guidewire. The winding creates an undulating surface that provides a tactile sensation for a user (e.g., clinician, operator, etc.) that is directing the guidewire. The tactile sensation is created by friction between the fingers of the user and the guidewire and allow for complex manipulations of the guidewire as it is directed through the tortuous path.

FIG. 1 is a diagram of a guidewire 100, according to an exemplary embodiment. The guidewire 100 includes a tip region 102, a transition region 104, and a proximal region 106. In some embodiments the tip region 102, the transition region 104, and the proximal region 106 are a unitary body. The tip region 102, the transition region 104, and the proximal region 106 can also be separate components that are joined together mechanically (e.g., welding, soldering, etc.) and/or chemically (e.g., via adhesives, etc.). The tip region 102 includes a tip wire 110, the transition region 104 includes a transition wire 112, and the proximal region 106 includes a proximal wire 114. In some embodiments, the tip wire 110, the transition wire 112, and the proximal wire 114 are constructed from the same material. The tip wire 110, the transition wire 112, and the proximal wire 114 can also be constructed from different materials. Each of the tip wire 110, the transition wire 112, and the proximal wire 114 can include one or more of a metal (e.g, stainless steel, nitinol, etc.) and a polymer (e.g., silicone, polytetrafluoroethylene, etc.). The tip wire 110, the transition wire 112, and the proximal wire 114 each have circular cross-sectional shapes, with the proximal wire 114 having a larger diameter than the tip wire 110. For example, the tip wire 110 may have a diameter between approximately 0.254 millimeters (“mm”), between approximately 0.381 mm and 0.4826 mm, or approximately 0.4572 mm. The proximal wire 114 may have a diameter between approximately 0.762 mm and 1.016 mm, between approximately 0.8128 mm and 0.9652 mm, or approximately 0.889 mm. As used herein, the term “approximately” refers to plus or minus ten percent. The transition wire 112 has a diameter that changes along its length such that, at the distal end of the transition wire 112, the diameter of the transition wire 112 is the same as the diameter of the tip wire 110, and at the proximal end of the transition wire 112, the diameter of the transition wire 112 is the same as the diameter of the proximal wire 114. As used herein, the term “distal” means further from the user (e.g., the “working end”) and the term “proximal” means closer to the user (e.g., the “handle end”). In some embodiments, the diameter of the transition wire 112 changes constantly along its length such that the transition between the tip wire 110 and the proximal wire 114 is smooth (e.g., linear). The diameter of the transition wire 112 can also change in a non-constant fashion (e.g., the transition wire 112 can have one or more step changes along its length such that the transition between the tip wire 110 and the proximal wire 114 is not smooth).

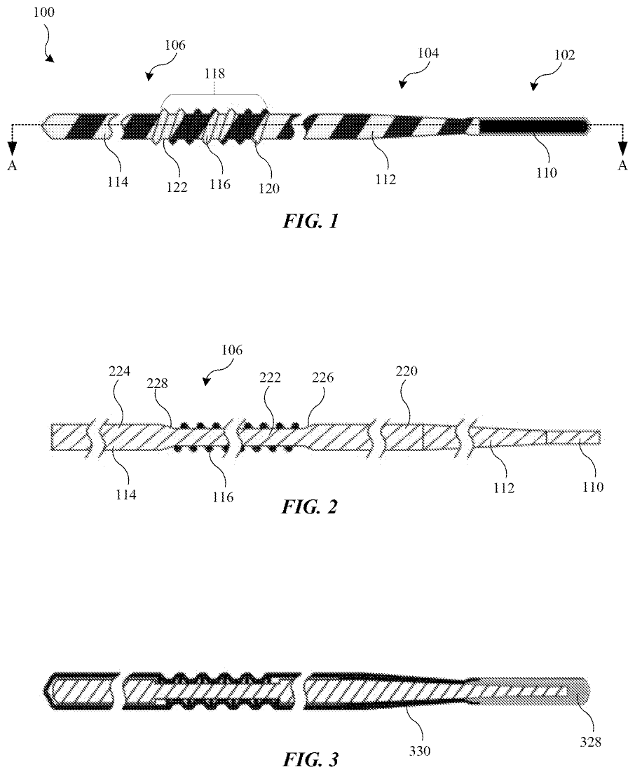

The proximal wire 114 is shown to include a winding 116. The winding 116 is coupled with the proximal wire 114 and extends radially from the proximal wire 114 such that the winding 116 extends radially beyond an outer surface of the proximal wire 114. As shown, the winding 116 extends around the proximal wire 114 in a helical manner. In some embodiments, the winding 116 covers the entire length of the proximal wire 114. The winding 116 can also cover a portion of the proximal wire 114 and leave a remaining portion of the proximal wire 114 uncovered (e.g., without the winding 116 extending radially therefrom). Arranged as described, the winding 116 defines an undulating surface 118 along at least a portion of the length of the proximal wire 114, where peaks 120 of the undulating surface 118 are defined by the winding 116 and valleys 122 of the undulating surface 118 are defined by the outer surface of the proximal wire 114. The peaks 120 and valleys 122 create corrugations (e.g., ribs) that provide a larger surface area for a user to grip than a guidewire with no winding. The larger surface area provides for more contact between the fingers of the user and the guidewire 100 than a guidewire with no windings, thereby causing more friction between the fingers of the user and the guidewire 100 than a guidewire with no windings. Accordingly, the user has more control with the guidewire 100 than if a guidewire with no winding were used. For example, the user can exert less force on the guidewire 100 when manipulating the guidewire 100 (e.g., pushing, turning, bending, twisting, knuckling, etc.) to reach the target site than if a guidewire with no winding were used. The winding 116 will be further described with reference to FIGS. 2-5.

FIG. 2 is a diagram of a cross-section of the guidewire 100 of FIG. 1 taken along the line A-A. As shown, the proximal wire 114 includes a first wire 220, a second wire 222, a remainder wire 224, a transition wire 226, and a transition wire 228. As shown, the second wire 222 has a smaller diameter than the first wire 220 and the remainder wire 224. The transition wire 226 couples the first wire 220 to the second wire 222 and the transition wire 228 couples the remainder wire 224 to the second wire 222 such that the transition wire 226 and the transition wire 228 provide for the change in diameters. In some embodiments, the geometry of the transition wire 226 and the transition wire 228 are the same (e.g., both include a ramp with a constant slope, a stepped portion, a concave portion, etc.). The geometry of the transition wire 226 and the transition wire 228 can also differ such that one of the transition wire 226 and the transition wire 228 includes a first geometry (e.g., a ramp with a constant slope, a stepped portion, a concave portion, etc.) and the other of the transition wire 226 and the transition wire 228 includes a second geometry.

As shown, the winding 116 is coupled to the second wire 222 such that a bottom surface of the winding 116 is recessed radially relative to an outer surface of the first wire 220 and the remainder wire 224. The top surface of the winding 116 extends radially from the outer surface of the first wire 220 such that the top surface of the winding 116 extends radially beyond the outer surface of the first wire 220 and the remainder wire 224. Accordingly, even though the winding 116 is partially recessed, the winding 116 still creates the undulating surface 118 as described with reference to FIG. 1.

In some embodiments, an outer diameter of the second wire 222 is the same as that of the first wire 220 and the remainder wire 224 such that the first wire 220, the second wire 222, and the remainder wire 224 are a single wire with no transitions (e.g., no transition wire 226 and no transition wire 228). In such embodiments, the winding 116 still creates the undulating surface 118.

FIG. 3 is another diagram of a cross-section of the guidewire 100 of FIG. 1 FIG. 1 taken along the line A-A. As shown, the guidewire 100 includes a tip tubing 328 and a cover 330. The tip tubing 328 is a polymer tube coupled to the tip wire 110. The tip tubing 328 is added so that it can contact gastrointestinal structures as the guidewire 100 is navigated through a tortuous path without causing damage to the structures. The cover 330 is configured to enclose the components of the guidewire 100. In some embodiments, the cover 330 is a polymer tube that shrinks around the guidewire 100 such that the components of the guidewire 100 are enclosed within the cover 330.

In some embodiments, the cover 330 covers the tip tubing 328 after the tip tubing 328 is assembled to the tip wire 110. The cover 330 can also be coupled with the tip tubing 328 after the tip tubing 328 is assembled to the tip wire 110 (e.g., with adhesive, etc.).

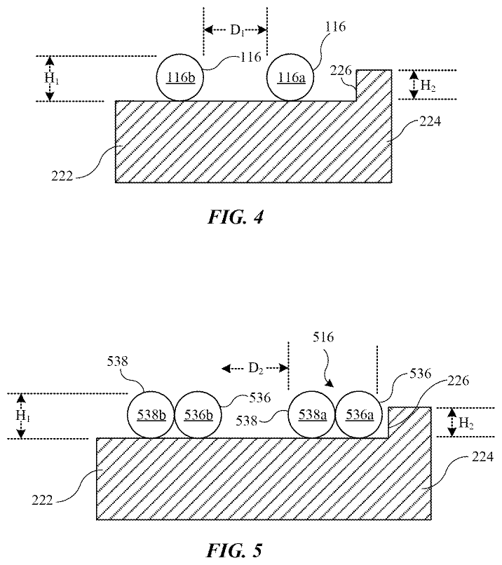

FIG. 4 is a diagram of the winding 116 of the guidewire 100 of FIG. 1. The winding 116 is shown to include a first coil 116a and a second coil 116b, where a coil of the winding 116 is defined as a portion of the winding 116 that extends around the second wire 222 for one revolution. For example, the first coil 116a begins in the position as shown in FIG. 4, extends around the second wire 222 for an entire revolution, and meets the second coil 116b where the second coil 116b begins (e.g., in the position shown in FIG. 4). Accordingly, though only two coils are shown of the winding 116, a plurality of coils may be used (e.g., two coils, ten coils, fifty coils, one-hundred coils, etc.). As shown in FIG. 4, the transition wire 226 is shown as a step between the first wire 220 and the second wire 222. As described, this is an example configuration and the transition wire 226 can include a variety of configurations (e.g., an angled slope between first wire 220 and second wire 222, a concave portion between first wire 220 and second wire 222, a convex portion between first wire 220 and second wire 222, etc.).

As shown, the outer surface of first wire 220 extends radially beyond the outer surface of the second wire 222 by a height of H₂. Each of the first coil 116a and the second coil 116b extends beyond the outer surface of second wire 222 by a height of H₁, where H₁ is equal to the cross-sectional diameter of the winding 116. In some embodiments, H₁ is at least 0.01 mm. In some embodiments, H₁ is at least 0.05 mm, or at least 0.07 mm, or at least 0.10 mm. Accordingly, in some embodiments the winding 116 extends radially beyond the outer surface of the first wire 220 by a height of H₁-H₂. As described above, in some implementations the outer diameters of the first wire 220, the second wire 222, and the remainder wire 224 are the same (e.g., the first wire 220, the second wire 222, and the remainder wire 224 may be a unitary component). In such implementations, H₂ is equal to zero and the winding 116 extends radially beyond the outer surfaces of the first wire 220, the second wire 222, and the remainder wire 224 by a height of H₁.

In addition, a distance between the first coil 116a and the second coil 116b is defined as D₁. D₁ is arranged such that a finger of the user contacts at least the first coil 116a and the second coil 116b when manipulating the guidewire 100. For example, there is a maximum threshold distance over which a finger of a user can fit between the first coil 116a and the second coil 116b such that the user perceives the guidewire 100 as having no undulating surface 118. Accordingly, D₁ is at most equal to the maximum threshold distance. In some embodiments, the maximum threshold distance is twenty mm. The maximum threshold distance can also be, for example, fifteen mm, ten mm, five mm, four mm, etc. Furthermore, D₁ is arranged such that the user can distinguish between the first coil 116a and the second coil 116b when manipulating the guidewire 100. For example, as D₁ becomes smaller there is a minimum threshold distance under which the user cannot determine that there are multiple surfaces (e.g., the user perceives the guidewire as having no undulating surface 118). Accordingly, D₁ is at least equal to the minimum threshold distance. In some embodiments, the minimum threshold distance is 0.5 mm. The minimum threshold distance can also be, for example, 0.6 mm, 0.7 mm, 0.8 mm, 0.9 mm, or 1.0 mm.

As shown, the first coil 116a and the second coil 116b of the winding 116 have a circular cross-section. In some embodiments, the winding 116 can have different cross-sectional shapes. For example, the winding 116 can have a cross-sectional shape including an ellipse, a square, a pentagon, a hexagon, an octagon, or any other geometric shape that would provide the functionality described herein.

FIG. 5 is a diagram of another winding 516, according to an exemplary embodiment. The winding 516 includes a first winding 536 and a second winding 538, where each of the first winding 536 and the second winding 538 are similar to the winding 116. The first winding 536 includes a first coil 536a and a second coil 536b, and the second winding 538 includes a first coil 538a and a second coil 538b. In some embodiments, the first winding 536 and the second winding 538 are coupled together prior to being coupled to the second wire 222. The first winding 536 and the second winding 538 can also be separate components that are coupled to the second wire 222 during the manufacturing process. As shown, the first winding 536 and the second winding 538 have the same cross-sectional shape and extend radially beyond the outer surface of the second wire 222 to the same height, H₁. In some embodiments, the first winding 536 and the second winding 538 may include different cross-sectional shapes. For example, the first winding 536 may include a circular cross-sectional shape and the second winding 538 may include a square cross-sectional shape. Furthermore, in some embodiments the first winding 536 and the second winding 538 may extend radially beyond the outer surface of the second wire 222 to different heights. For example, the first winding 536 may extend radially beyond the outer surface of second wire 222 to the height H₁, and the second winding 538 may extend radially beyond the outer surface of the second wire 222 to another height (not shown) that is greater than or less than H₁.

As described with reference FIG. 4, H₁ is at least 0.01 mm. In some embodiments, H₁ is at least 0.05 mm, or at least 0.07 mm, or at least 0.10 mm. Accordingly, in some embodiments the first winding 536 and the second winding 538 extend radially beyond the outer surface of the first wire 220 by a height of H₁-H₂. Furthermore, a distance D₂ between the first coil 538a and the second coil 536b is less than the maximum threshold distance (e.g., twenty mm) and greater than the minimum threshold distance (e.g., 0.5 mm) as described with reference to FIG. 4. In embodiments where the first winding 536 and the second winding 538 are separate components, the first winding 536 and the second winding 538 may define a gap therebetween, where the gap is less than the distance D₂.

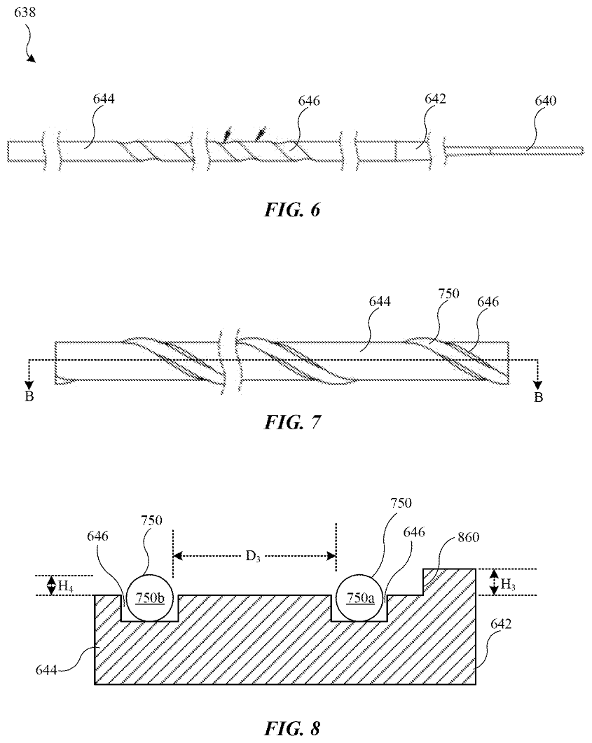

FIGS. 6-7 are diagrams of another guidewire 638, according to an exemplary embodiment. As shown, the guidewire 638 includes a tip wire 640, a transition wire 642, and a proximal wire 644 that defines a recess 646. In some embodiments, the tip wire 640, the transition wire 642, and the proximal wire 644 are constructed from the same material. The tip wire 110, the transition wire 112, and the proximal wire 114 can also be constructed from different materials. Each of the tip wire 640, the transition wire 642, and the proximal wire 644 can include one or more of a metal (e.g, stainless steel, nitinol, etc.) and a polymer (e.g., silicone, polytetrafluoroethylene, etc.). The tip wire 640, the transition wire 642, and the proximal wire 644 each have circular cross-sectional shapes, with the proximal wire 644 having a larger diameter than the tip wire 640. The transition wire 642 has a diameter that changes along its length such that, at the distal end of the transition wire 642, the diameter of the transition wire 642 is the same as the diameter of the tip wire 640, and at the proximal end of the transition wire 642, the diameter of the transition wire 642 is the same as the diameter of the proximal wire 644. In some embodiments, the diameter of the transition wire 642 changes constantly along its length such that the transition between the tip wire 640 and the proximal wire 644 is smooth (e.g., linear). The diameter of the transition wire 642 can also change in a non-constant fashion (e.g., the transition wire 642 can have one or more step changes along its length such that the transition between the tip wire 640 and the proximal wire 644 is not smooth).

The proximal wire 644 defines the recess 646 that extends into and helically around the proximal wire 644. The recess 646 is configured to receive a winding 750. The winding 750 is similar to the winding 116 such that the description of the winding 116 pertains to the winding 750.

FIG. 8 is a diagram of a cross-section of a portion of the guidewire 638 of FIG. 7 taken across the line B-B. As shown, the winding 750 includes a first coil 750a and a second coil 750b, where a coil of the winding 750 is defined as a portion of the winding 750 that extends around the proximal wire 644 for one revolution. For example, the first coil 750a begins in the position as shown in FIG. 8, extends around the proximal wire 644 for an entire revolution, and meets the second coil 750b where the second coil 750b begins (e.g., in the position shown in FIG. 8). Accordingly, though only two coils are shown of the winding 750, a plurality of coils may be used (e.g., two coils, ten coils, fifty coils, one-hundred coils, etc.). As shown in FIG. 8, the transition wire 642 is coupled to the proximal wire 644 at a transition region 860. The transition region 860 is shown is shown to include a step between the transition wire 642 and the proximal wire 644. The transition region 860 can also include a variety of configurations (e.g., an angled slope between the transition wire 642 and the proximal wire 644, a concave portion between the transition wire 642 and the proximal wire 644, a convex portion between the transition wire 642 and the proximal wire 644, etc.). As shown, the transition region 860 extends radially beyond an outer surface of the proximal wire 644 by a height of H₃. In some embodiments, there is no transition region such that H₃=0 (e.g., the outer surface of the transition wire 642 and the outer surface of proximal wire 644 are positioned at the same radial distance from a longitudinal axis that extends through the center of the guidewire 638).

Each of the first coil 750a and the second coil 750b extends beyond the outer surface of proximal wire 644 by a height of H₄. In some embodiments, H₄ is at least 0.01 mm. In some embodiments, H₄ is greater than zero and less than the diameter of the winding 750. In addition, a distance between the first coil 750a and the second coil 750b is defined as D₃. D₃ is arranged such that a finger of the user contacts at least the first coil 750a and the second coil 750b when manipulating the guidewire 638. For example, there is a maximum threshold distance over which a finger of a user can fit between the first coil 750a and the second coil 750b such that the user perceives the guidewire 638 as having no undulating surface. Accordingly, D₃ is at most equal to the maximum threshold distance. In some embodiments, the maximum threshold distance is twenty mm. The maximum threshold distance can also be, for example, fifteen mm, ten mm, five mm, four mm, etc. Furthermore, D₃ is arranged such that the user can distinguish between the first coil 750a and the second coil 750b when manipulating the guidewire 638. For example, as D₃ becomes smaller there is a minimum threshold distance under which the user cannot determine that there are multiple surfaces (e.g., the user perceives the guidewire as having no undulating surface). Accordingly, D₃ is at least equal to the minimum threshold distance. In some embodiments, the minimum threshold distance is 0.5 mm. The minimum threshold distance can also be, for example, 0.6 mm, 0.7 mm, 0.8 mm, 0.9 mm, or 1.0 mm.

As shown, the first coil 750a and the second coil 750b of the winding 750 have a circular cross-section. In some embodiments, the winding 750 can have different cross-sectional shapes. For example, the winding 750 can have a cross-sectional shape including an ellipse, a square, a pentagon, a hexagon, an octagon, or any other geometric shape that would provide the functionality described above.

Furthermore, the recess 646 is shown as having a rectangular cross-section. In some embodiments, the recess 646 can have different cross-sectional shapes. For example, the recess 646 can have a cross-sectional shape including an ellipse, a square, a pentagon, a hexagon, an octagon, or any other geometric shape.

As shown, the cross-sectional shapes of the winding 750 and the recess 646 are different. In some embodiments, the cross-sectional shapes of the winding 750 and the recess 646 are the same. For example, the cross-sectional shape of the winding 750 can be a square, and the cross-sectional shape of the recess 646 can be a square.

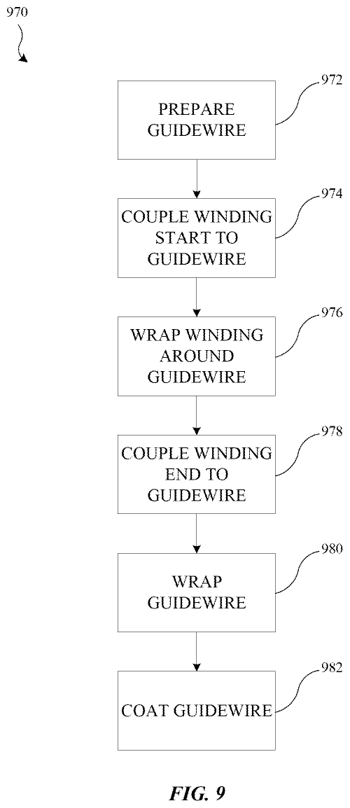

FIG. 9 is a flow chart of a process 970 to manufacture a guidewire, according to an exemplary embodiment.

At operation 972, the guidewire is prepared to receive a winding. For example, and with reference to FIGS. 1-5, the second wire 222 may be treated to facilitate coupling with the winding 116. For example, the second wire 222 may be roughened, smoothed, cleaned, etc., to ensure a strong connection between the second wire 222 and the winding 116. With reference to FIGS. 6-8, the recess 646 may be cut into the proximal wire 644, and the recess 646 may be treated as described to ensure a strong connection between the proximal wire 644 and the winding 750.

At operation 974, the winding start is coupled to the guidewire. For example, the winding 116 and the winding 750 each include a distal end (e.g., the winding start) that is coupled to the second wire 222 and the proximal wire 644, respectively. Coupling can be achieved via mechanical processes such as welding, soldering, brazing, etc. Coupling can also be achieved via chemical processes like adhesive bonding.

At operation 976, the winding is wrapped around the guidewire. For example, the winding 116 is wrapped around the second wire 222 so as to maintain D₁ above the minimum threshold distance and below the maximum threshold distance. In some embodiments, the winding 116 is coupled to the second wire 222 at various positions along the length of the second wire 222 such that D₁ remains substantially the same (e.g., within ten percent) along the length of the second wire 222. As another example, the winding 750 is wrapped around the proximal wire 644 such that the winding 750 remains within the recess 646. Accordingly, it may not be necessary to couple the winding 750 to the proximal wire 644 at various locations along the length of the proximal wire 644 as the recess 646 defines the spacing D₃ between coils of the winding 750.

At operation 978, the winding end is coupled to the guidewire. For example, the winding 116 and the winding 750 each include a proximal end (e.g., the winding end) that is coupled to the second wire 222 and the proximal wire 644, respectively. Coupling can be achieved via mechanical processes such as welding, soldering, brazing, etc. Coupling can also be achieved via chemical processes like adhesive bonding. In some instances, the winding end is coupled to the guidewire at the proximal end of the guidewire such that the proximal end of the guidewire is covered by the winding. In some embodiments, the winding end is coupled to the guidewire such that a remainder of the guidewire is not covered by the winding.

At operation 980, the guidewire is wrapped. As described above, the guidewire (e.g., the guidewire 100 or the guidewire 638) is covered to protect the components of the guidewire. For example, the guidewire may be inserted through a piece of shrink tubing, and the shrink tubing may be heated up such that the shrink tubing shrinks around the guidewire, thereby maintaining the position of the winding 116 and the winding 750 on the second wire 222 and the proximal wire 644, respectively.

At operation 982, the guidewire is coated. In some instance, a lubricious coating is applied to the guidewire to facilitate insertion into and navigation through tortuous gastrointestinal structures. In some embodiments, the coating is applied just to the tip of the guidewire (e.g., the tip wire 110 and the tip wire 640). The coating may also be applied to the entire guidewire or a portion thereof.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A guidewire, comprising:
a wire that includes an outer surface that defines a diameter; and
a winding that extends around the wire and defines an undulating surface that extends along a length of the wire;
wherein a peak of the undulating surface is defined by the winding and a valley of the undulating surface is defined by the wire.

2. The guidewire of claim 1, further comprising a wrap that covers the winding and the wire.

3. The guidewire of claim 1, wherein the winding extends radially beyond the outer surface by at least 0.01 millimeters.

4. The guidewire of claim 1, wherein the wire includes a first wire and a second wire;
wherein the first wire includes the outer surface that defines the diameter and the second wire includes an additional outer surface that defines an additional diameter that is smaller than the diameter; and
wherein the winding extends around the second wire and the peak of the undulating surface extends radially from the outer surface.

5. The guidewire of claim 1, wherein the winding includes a first coil and a second coil, and a distance between the first coil and the second coil is less than a threshold value.

6. The guidewire of claim 5, wherein the threshold value is twenty millimeters.

7. The guidewire of claim 1, wherein the winding has a cross-sectional shape including one or more of a circle, an ellipse, a square, a pentagon, a hexagon, and an octagon.

8. A guidewire, comprising:
a wire that includes an outer surface that defines a diameter, wherein the wire defines a recess extending into and helically around the wire; and
a winding configured to fit within the recess and extend helically around the wire.

9. The guidewire of claim 8, further comprising a wrap that covers the winding.

10. The guidewire of claim 8, wherein the winding extends radially beyond the outer surface of the wire by at least 0.01 millimeters.

11. The guidewire of claim 10, wherein the winding extends radially beyond the outer surface of the wire by at least 0.07 millimeters.

12. The guidewire of claim 8, wherein the winding has a cross-sectional shape including one or more of a circle, an ellipse, a square, a pentagon, a hexagon, and an octagon.

13. The guidewire of claim 8, the winding includes a first coil and a second coil, and a distance between the first coil and the second coil is less than a threshold value.

14. A method of producing a guidewire, comprising:
coupling a first end of a winding to a wire;
wrapping the winding around the wire; and
coupling a second end of the winding to the wire.

15. The method of claim 14, further comprising:
removing material from the wire in a helical fashion to create a helical recess extending around the wire; and
coupling the first end of the winding to the wire in the helical recess;
wrapping the winding around the wire in the helical recess; and
coupling the second end of the winding to the wire in the helical recess.

16. The method of claim 14, wherein wrapping the winding around the wire includes covering a portion of the wire with the winding and leaving a remainder of the wire uncovered.

17. The method of claim 14, wherein the winding extends radially beyond an outer surface of the wire by at least 0.01 millimeters.

18. The method of claim 17, wherein the winding extends radially beyond the outer surface of the wire by at least 0.07 millimeters.

19. The method of claim 16, wherein the remainder of the wire is at least 0.05 millimeters in length.

20. The method of claim 14, wherein the winding has a cross-sectional shape including one or more of a circle, an ellipse, a square, a pentagon, a hexagon, and an octagon.
